(19) United States
(12) Patent Application Publication
(10) Pub. No.: US 2004/0132123 A1
(43) Pub. Date: Jul. 8, 2004

(54) Methods and kits utilizing heparanase as a diagnostic marker for haemostatic disorders

(76) Inventor: Oron Yacoby-Zeevi, Moshav Bizaron (IL)

Correspondence Address: Anthony Castorina, G E Ehrlich, Suite 207, 2001Jefferson Davis Highway, Arlington, VA, 22202, US

(21) Appl. No.: 10/475,591

(22) PCT Filed: May 9, 2002
(86) PCT No.: PCT/IL02/00362

(51) Int. Cl. G01N 33/53; G01N 33/537; G01N 33/543

(52) U.S. Cl. 435/7.92

(57) ABSTRACT

Provided is a method of determining a presence, absence, or severity of a haemostatic disorder in a subject. The method is effected by determining a level of heparanase expression or activity in a biological sample obtained from the subject. Also provided are kits for use with the method.

### FIELD AND BACKGROUND OF THE INVENTION

The present invention relates to methods and kits, which can be used to diagnose haemostatic disorders. More specifically, the present invention relates to methods and kits, which utilize heparanase as a diagnostic marker for haemostatic disorders.

Haemostatic (or hemostatic) disorders encompass a variety of disease conditions associated with an interruption of the flow of blood due to coagulation. Blood coagulation is a complex process of interaction between various blood factors, primarily platelets and fibrin and other components, which eventually give rise to a clot (thrombus). Generally, a coagulation “cascade” starts with an activation of proteolytic enzymes (proenzymes or zymogens in their inactive form) by an activated clotting factor, which ends in a clot formation by fibrin, platelets and numerous other blood components. An activated fibrin-bound thrombin cleaves fibrinogen and activates platelets, and thus facilitates continuous growth of the clot.

The wall of blood vessels play a major role both in protecting against, and in promoting, thrombosis [Hirsh J, Weitz JI; Semin Hematol 1999; 36(4 Suppl 7):118-32]. More particularly, the surface-bound heparan sulfate (HS) endows blood vessels with an anticoagulant property [Scully MF et al; FEBS Lett, 1988, 241(1-2): 11-4].

In primary haemostasis, activated platelets interact with heparan sulfate which is bound to the surface of blood vessels. Platelets are activated by exposed subendothelial collagen or thrombin in an ordered sequence of events that includes shape change, increase in cytoplasmic Ca²⁺, activation of the alphaIIb beta3 integrin, granule secretion, aggregation, and formation of a stable haemostatic plug. Primary haemostasis may progress into a serious haemostatic disorder.

Haemostatic disorders encompass a variety of disease conditions associated with an interruption of the flow of blood due to coagulation or thrombosis. For example, thrombosis, which can complicate rupture of an atherosclerotic plaque, can cause a partial or a total occlusion of the affected blood vessel, thereby leading to a number of important cardiovascular complications, including unstable angina, acute myocardial infraction (heart attack), cerebral vascular accident (stroke), myocardial ischemia, arterial aneurisms, atherosclerosis, peripheral arterial disease, stent thrombosis and exercise induced ischemia. Vessel injury and/or stasis, posttraumatic or following an elective surgery, can trigger venous thrombosis causing deep vein thrombosis (DVT), pulmonary embolism (PE), cerebral ischemia and ischemia and reperfusion injury. Secondary hypercoagulable states may appear during malignancy, pregnancy, use of oral contraceptives, myeloproliferative disorders, hyperlipidemia, homocystinuria and lead to thrombotic events.

Autoimmune reactions such as heparin-induced thrombocytopenia and thrombosis (HITT), anti-phospholipid syndrome (APS), anti-cardiolipin sydrome and lupus can cause thrombotic tendencies. Inherited defects such as antithrombin III deficiency, protein C deficiency, decreased plasminogen activity, defective plasminogen activator, hyperthromocysteinemia, dysfibrogenemia and resistance to activated protein Ca can also increase the tendency to thrombosis.

Haemostatic disorders are a major cause of disability and mortality throughout the world and particularly in Western society.

Accurate diagnosis and monitoring of haemostatic disorders is critical for the appropriate treatment of patients. Patients suffering from a severe haemostatic disorder, or patients undergoing procedures prone to result in a haemostatic disorder, must be treated with very high doses of anti-coagulant or anti-thrombin drugs. Several such drugs have been developed, including heparin [Hirsh, Circ. 88:I-C (1993)], low molecular weight heparins [LMWHs; Boneu, et al., Thrombosis Research 40:81-89 (1985)], hirudin (U.S. Pat. Nos. 5,240,913 and 5,196,404), agratroban and PPACK (D-phenylalanyl-L-propyl-L-arginyl chloromethyl ketone). Platelet aggregation inhibitors such as aspirin, ticlopidine dipyridamole, clopidrogel, decorsin, thromboxane synthase inhibitors and receptor blockers are also used for treatment.

Moreover, numerous other anti-coagulant agents have been described in the literature. For instance, hirudin derivatives for blocking the active site of thrombin are described in U.S. Pat. Nos. 5,240,913 and 5,196,404. A bifunctional anti-thrombotic composition which includes both a glycoprotein IIb/IIIa inhibitory domain and a thrombin inhibitory domain is described in WO 92/10575. Peptide analogs of glycoprotein IIIa for thrombogenesis inhibition are described in WO 90/00178. Inhibitors of factor X and/or Xa are described in U.S. Pat. Nos. 5,239,058 and 5,189,019, and PCT publications WO 93/09803, WO 92/04378 and WO 92/01464. Inhibitors of factors VII and/or VIII are described in U.S. Pat. Nos. 5,223,427 and 5,023,236 and WO 92/06711. Platelet anti-adhesives and related antibodies are described in WO 92/08472. In addition, numerous modified heparin compositions, as well as other glycosaminoglycans and derivatives, have been developed. For example, U.S. Pat. Nos. 5,296,471, 5,280,016 and 5,314,876. More recently, various dermatan sulfates have been developed and their interactions with heparin cofactor II studied [Mascellani, et al., Thrombosis Research 74:605-615 (1994)]. Danaparoid sodium is a heparinoid glycosaminoglycuronan antithrombotic agent, which inhibits factors Xa and IIa at a ratio greater than heparin, with minimal effect on platelet function [Acostamadiedo JM et al; Expert Opin Pharmacol. 2000 1(4):803-14].

Although many anti-coagulant drugs have been developed, administrating such drugs to patients always carries a serious risk of an excessive bleeding, which is often life threatening. Therefore the selection of drug and its clinical use must be determined and monitored with the outmost caution and precision. Furthermore, evaluating the efficacy of drugs administered to patients, and monitoring the progress of patients is imperative.

Thus, diagnosing haemostatic disorders is critical for determining an appropriate treatment, either prophylactic or therapeutic. For example, pulmonary embolism condition which affect about 500,000 people annually in the U.S.A. alone, is associated with a mortality rate of 30% if undiagnosed and only 10% if diagnosed and appropriately treated. Likewise, monitoring haemostatic disorders is critical for evaluating efficacy of treatment, and for minimizing risky side effects.

The importance of accurately diagnosing and monitoring haemostatic disorders has led to the development of several diagnostic methods and to the intensive pursuit of still additional diagnostic approaches.

The contrast venogram is the standard for diagnosing deep venous thrombosis (DVT). This is an invasive procedure associated with few, but significant complications, including thrombosis, renal failure, allergic reactions, and anaphylactic shock.

Another approach that has become increasingly accepted for diagnosing DVT is venous ultrasound. Ultrasound procedures such as duplex Doppler ultrasonography have comparable sensitivity and specificity to the contrast venogram. However, because specialized equipment is required for conducting ultrasonography, there are often significant logistic and temporal difficulties in using this procedure for diagnosing DVT in emergency situations.

A popular noninvasive method of diagnosing DVT is based on the expression of D-dimer, which is a fragment of the cross-linked fibrin molecule released during plasmin-mediated clot lysis. The level of D-dimer in a sample can be measured using an antibody, either quantitatively by enzyme immunoassay, or qualitatively by latex agglutination assay. Hence, the D-dimer immunoassay has been used in clinical practice for diagnosing DVT and pulmonary embolism (PE). Several investigations over the past 5 years have evaluated the utility of a D-dimer assay to diagnose DVT. Unfortunately, a variety of conditions can independently elevate D-dimer in the absence of thrombosis, thus leading to false positive diagnosis. These conditions include age, sepsis, malignancy, trauma, congestive heart failure, uremia, stroke, and myocardial infection. Hence, the unacceptably low specificity in almost all of these studies makes this a poor screening test for clinical diagnosis of DVT [Kozman H et al; South Med. J. 1997, 90(9):907-10].

Another noninvasive test for diagnosing haemostatic disorders is described by Jaffe AS et al [J Am Coll Cardiol. 1984, 4(4): 653-9]. This test is based on platelet factor IV (PF4) and beta-thromboglobulin, which are protein constituents of platelet granules. Elevated levels of these proteins in plasma have been used as sensitive indicators of platelet degranulation. While increases of PF4 have been observed in samples taken from patients with infarction, the implication that they reflect pathogenetic phenomena, such as coronary thrombosis, has not been assessed explicitly. Furtheremore, PF4 values generally remain normal despite acute myocardial infarction. Rare increases that occur reflect platelet degranulation in vitro due to sampling artifact, or perturbations of platelets in vivo due to invasive procedures. Hence, the PF4 test is not sufficiently reliable.

A radioimmunoassay described by Wu g et al [Nouv Rev Fr Hematol. 1992, 34(1): 31-5] utilizes an antibody specific for an alpha-granule membrane protein that associates with the platelet surface during secretion. Quantification of their binding by flow cytometry allows an estimation of epitope expression within the whole platelet population. However, tests failed to show a direct correlation between the presence of these epitopes and future pathological events such as thrombosis and cardiovascular diseases. [Nurden AT et al; Nouv Rev Fr Hematol. 1993, 35(1): 67-71]. Another anti platelet activation-dependent granule-external membrane protein (PADGEM) may be utilized to visualize thrombi in radioimmunoscintigraphy [Palabrica TM et al; PNAS 1989, 86(3): 1036-40]. In general, it is difficult to detect platelet membrane changes that accompany in vivo activation if only a small fraction of circulating platelets has undergone secretion. Hence, this test appears to be lacking the sensitivity required for accurately diagnosing haemostatic disorders.

U.S. Pat. No. 5,814,462 describes a noninvasive method for identifying the presence or absence of ischemia. The method is based on a fibroblast growth factor (bFGF) and an endothelial growth factor (VEGF) as biochemical markers. The method is selective for detecting ischemia, thus not applicable for use in diagnosing other haemostatic disorders.

U.S. patent application Ser No. 09/860,618 describes a method for diagnosing haemostatic disorders by determining the expression of P-selectin in a blood sample. More recently, an immunoassay kit was introduced by American Biogenetic Sciences Inc., which is based on a thrombus-precursor protein as a marker for thrombosis. Another immunoassay kit has been recently introduced by Corgenix Medical Corporation, which is based on anti-prothrombin markers for detecting cardiovascular haemostatic disorders.

There is thus a widely recognized need for, and it would be highly advantageous to have a method and a kit which can be easily utilized to diagnose a wide variety of haemostatic disorders in clinical practice.

The present invention successfully addresses the shortcomings of the presently known configurations by providing a method and kit which can be used to accurately diagnose a haemostatic disorder or condition in an individual.

### SUMMARY OF THE INVENTION

It is a principle object of this invention to provide a method and a kit for the detection and monitoring of haemostatic disorders which is noninvasive, broad-spectrum, selective, sensitive, quantitative, reliable, and simple to practice.

Thus, according to one aspect of the present invention there is provided a method of determining a presence, absence, or severity of a haemostatic disorder in a subject, the method comprising determining a level of heparanase expression or activity in a biological sample obtained from the subject and based on the level of the heparanase expression or activity determining the presence, absence, or severity of the of haemostatic disorder in the subject.

According to further features in preferred embodiments of the invention described below, the method further comprises comparing the level of heparanase expression or activity in the biological sample obtained from the subject to that of a normal individual and/or an individual suffering from the haemostatic disorder.

According to still further features in the described preferred embodiments the biological sample is a biological fluid selected from the group consisting of blood, plasma, cerebral fluid, and urine.

According to still further features in the described preferred embodiments the determining the level of heparanase expression in the biological sample is effected via an immunoassay.

According to still further features in the described preferred embodiments the immunoassay is selected from the group consisting of enzyme-linked immunosorbent assay (ELISA), a radioimmunoassay, an immunofluorescence assay, and a light emission immunoassay.

According to still further features in the described preferred embodiments the determining the level of heparanase activity in the biological sample is effected by using an heparanase substrate.

According to still further features in the described preferred embodiments the heparanase substrate is selected from the group consisting of heparan sulfate proteoglycans, heparan sulfate, heparin, and heparin-sepharose.

According to still further features in the described preferred embodiments the heparanase substrate includes a detectable moiety selected from the group consisting of a chromogenic moiety, a fluorogenic moiety and a light emitting moiety.

According to another aspect of the present invention there is provided a kit for diagnosing a haemostatic disorder comprising at least one container including at least one reagent for determining a level of heparanase expression or activity in a biological sample, and packaging material identifying the at least one reagent for use in diagnosing the haemostatic disorder.

According to still further features in the described preferred embodiments the reagent includes a heparanase specific antibody.

According to still further features in the described preferred embodiments the heparanase specific antibody is coupled to an enzyme.

According to still further features in the described preferred embodiments the reagent includes a substrate of the enzyme.

According to still further features in the described preferred embodiments the substrate includes a chromogenic, fluorogenic or light emitting moiety.

According to still further features in the described preferred embodiments the reagent includes a heparanase substrate.

According to still further features in the described preferred embodiments the heparanase substrate is selected from the group consisting of heparan sulfate proteoglycans, heparan sulfate, heparin, and heparin-sepharose.

According to still further features in the described preferred embodiments the heparanase substrate includes a detectable moiety selected from the group consisting of a chromogenic moiety, a fluorogenic moiety, and a light emitting moiety.

According to yet another aspect of the present invention there is provided a method of monitoring an effectiveness of a haemostatic disorder treatment, comprising determining a level of heparanase expression or activity in biological samples obtained from a subject prior to, during and/or following treatment for the haemostatic disorder and based on the level of the heparanase expression or activity, monitoring the effectiveness of the haemostatic disorder treatment.

According to still further features in the described preferred embodiments the haemostatic disorder treatment is effected by administering to the individual a pharmaceutical composition.

According to still further features in the described preferred embodiments the pharmaceutical composition includes an active ingredient selected from the group consisting of a thrombin inhibitor, an anticoagulant, a platelet aggregation inhibitor and a heparanase inhibitor.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates heparanase level measured in plasma and serum samples kept either at room temperature or frozen at −80° C.

### DESCRIPTION OF THE PREFERRED EMBODIMENTS

The present invention is of a method and a kit which utilize heparanase as a diagnostic marker for haemostatic disorders. Specifically, the methodology of the present invention can be used to determine the presence, absence or severity of haemostatic conditions, to assess risk of developing haemostatic disorders, and to monitor efficacy of prophylactic or therapeutic drugs or treatments.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The enzyme endo-β-D-glucuronidase (heparanase) is secreted in relatively large amounts by platelets upon activation and degrades heparan sulfate [Eldor A et al; Semin Thromb Hemost 1987, 13(4):475-88]. Heparan sulfate proteoglycans (HSPGs) bound to endothelial cells tether regulatory factors, such as antithrombin III and superoxide dismutase, which regulate normal vascular function. Therefore, the cleavage and release of surface-bound HS by heparanase may cause a substantial loss of endothelial cell anticoagulant properties [Ihrke NS et al; J Cell Physiol. 1998, 175(3): 255-67]. Hence, activated platelets and heparanase secreted from activated platelets are critical components of primary haemostasis.

In addition to their involvement in primary haemostasis, activated platelets are also involved in the pathogenesis of many haemostatic diseases, including atherosclerosis, stroke, and thrombosis [Covic L et al; Biochemistry 2000, 39(18): 5458-67]. Increased platelet activation state in bone marrow transplant recipients may contribute to the thrombotic phenomena observed in these patients [Neumeister P et al; Eur. J Med. Res. 1998, 3(10): 465-9]. Chronic myeloproliferative disorders (MPDs) are characterized by a high incidence of thrombohaemorrhagic complications, possibly caused by platelet dysfunction [Jensen MK et al; Br J Haematol 2000, 110(1):116-24]. Clearly, activated platelets are involved in the haemostatic disorder from the disease onset (primary haemostasis) and throughout the disease progression.

While reducing the present invention to practice, the present inventor has further investigated biochemical interactions and processes participating in the formation and progression of haemostatic conditions. This investigation has resulted in the surprising discovery that heparanase is an excellent diagnostic marker for haemostatic disorders. As illustrated in Example 3 of the Examples section which follows, plasma levels of heparanase were substantially higher in patients suffering from a haemostatic condition as compared to healthy individuals and patients suffering from other unrelated diseases. Furthermore, the present study also demonstrated that heparanase is secreted in relatively large amounts from the onset and throughout progression of a haemostatic condition and as such, the level of heparanase can also be used as a reliable marker for diagnosing the severity or state of haemostatic disorder.

Thus, according to one aspect of the present invention there is provided a method of determining a presence, absence or severity of a haemostatic disorder in a subject, such as a human.

As used herein the phrase “haemostatic disorder” refers to a variety of disease conditions associated with an interruption of the flow of blood due to coagulation or thrombosis.

Examples of haemostatic disorders include cardiovascular complications, including unstable angina, acute myocardial infraction (heart attack), cerebral vascular accident (stroke), myocardial ischemia, arterial aneurisms, atherosclerosis, peripheral arterial disease, stent thrombosis and exercise induced ischemia, venous thrombosis and deep vein thrombosis (DVT), pulmonary embolism (PE), ischemia, thromboembolism due to autoimmune reactions including heparin-induced thrombocytopenia and thrombosis (HITT), anti-phospholipid syndrome (APS), anti-cardiolipin sydrome and lupus, antithrombin III deficiency, protein C deficiency, decreased plasminogen activity, defective plasminogen activator, hyperthromocysteinemia, dysfibrogenemia and resistance to activated protein Ca, secondary hypercoagulable states due to lupus inhibitor and acquired antithrombin III and protein S deficiencies.

The method is effected by determining the level of heparanase expression or activity in a biological sample obtained from the subject. Preferably, the biological sample is a biological fluid such as for example, blood, serum, plasma, cerebral fluid or urine which is obtained form the subject using well known techniques.

The biological sample can be analyzed directly or alternatively it can be processed prior to analysis. Processing of the biological sample can include adding an anticoagulant (e.g., EDTA, heparin and citrate), an anti-protease or a preservative. The sample may be analyzed whole, centrifuged or fractionated. The sample may be analyzed fresh, refrigerated or frozen.

As mentioned herein above diagnosis of a haemostatic disorder according to the present invention can be effected by several methods designed for determining the level of heparanase expression or activity in a biological sample.

As used herein the phrase “heparanase expression” refers to transcription or transcription/translation of heparanase. Several methods can be utilized to determine the level of heparanase expression including, but not limited to, immunoassays, western blotting, RT-PCR, northern blotting, or cDNA arrays.

According to a preferred embodiment of this aspect of the present invention, determining the level of heparanase expression in the biological sample is effected via an immunoassay utilizing a heparanase specific antibody. Immunoassays are fully explained in, for example “Using antibodies: A Laboratory Manual” [Ed Harlow, David Lane eds., Cold Spring Harbor Laboratory Press (1999)] and those familiar with the art will be capable of implementing various immunoassay techniques for determining the level of heparanase expression or activity.

As used herein, the term “heparanase specific antibody” refers to any monoclonal or polyclonal immunoglobulin, or a fragment thereof (e.g. Fab), that specifically binds to heparanase. An example of a heparanase specific antibody is provided by U.S. Pat. No. 6,177,545.

Immunoassay techniques suited for use as part of the present invention include, but not limited to, an enzyme-linked immunosorbent assay (ELISA), radioimmunoassay (RIA), immunofluorescent assay, and a light emission immunoassay all of which are well known in the art.

It should be noted that further description of methodology, which can be used to determine heparanase expression levels, is provided in the Examples section which follows.

The Results described in the Examples section emphasize the feasibility of using a heparanase specific antibody to determine the level of heparanase expression in serum and plasma samples obtained from human subjects.

As illustrated in Examples 1 and 2, the level of heparanase expression, as determined by ELISA, substantially increases during blood coagulation events (primary haemostasis), which may indicate a predisposition or an onset of a haemostatic disorder/condition. As illustrated in Table 1 the mean level of plasma heparanase increases in response to thrombin (which induces coagulation) and the mean level of serum heparanase is higher than plasma heparanase by at least five fold.

Example 3 clearly illustrates the feasibility of using heparanase as a reliable diagnostic marker to determine the presence or absence of haemostatic disorders in human patients. As shown in Table 2, the level of plasma heparanase in normal healthy people was established to be less than 150 ng/ml. As illustrated in Table 3 the level of plasma heparanase in samples obtained from all 15 patients (100%) diagnosed with thrombotic disorders exceeded 150 ng/ml, hence no false positive was diagnosed. Furthermore, among 23 patients which had plasma heparanase levels not higher than 150 ng/ml, all but one were not diagnosed with thromboembolic disorders.

The methodology of determining the level of heparanase expression may benefit the clinical practice in several ways including, but not limited to, diagnosing presence or absence of haemostatic disorders, or monitoring disease severity.

Diagnosis of haemostatic disorder according to the present invention can be effected by comparing the level of heparanase expression of a tested individual to that of one or more control subjects with a normal haemostatic activity. Whenever the level of heparanase expression or activity of the test subject is determined as being higher (preferably statistically higher) than the level of heparanase expression or activity in the control(s), diagnosis is indicated as positive. On the other hand, whenever the level of heparanase expression or activity in the test subject is determined as being substantially similar to the control(s), the diagnosis is indicated as negative.

Diagnosis according to the present invention may also involve setting standards of reference for normal or diseased individuals. Such standards may be differentiated among different control groups of people, characterized by gender, age groups, etc. Similarly, standards may be set for “progressive” haemostatic disorders, which may also be differentiated by different groups of people, characterized by the type and stage of haemostatic disorder, age, gender, etc. Hence, determining the severity of haemostatic disorders may be performed by comparative analysis of the level of heparanase in the sample obtained from a test subject with reference to established standards.

Since heparanase is an enzyme which catalyses heparan sulfate, analysis of heparanase catalytic activity can also be used by the method of the present invention in order to determine the presence absence or severity of a haemostatic disorder.

As used herein the term “heparanase activity” refers to the catalytic endoglycosidase activity of heparanase enzyme.

The level of heparanase activity in the biological sample can be determined using a heparanase specific substrate, such as, but not limited to, heparan sulfate proteoglycans, heparan sulfate, heparin, heparin-sepharose or derivatives. Further description of heparanase activity assays are provided in U.S. Pat. No. 6,190,875.

As used herein the term “heparanase specific substrate” refers to a substrate that can be cleaved by heparanase into products. Preferably the substrate is heparan sulfate, heparin, heparin-sepharose or derivative thereof.

To facilitate detection, the heparanase specific substrate preferably includes a detectable moiety such as, a chromogenic moiety (e.g., indoxyl, ONP, PNP, TMB), a fluorogenic moiety (e.g., methylumbelliferyl, methylcoumarin) or a light emitting moiety (e.g., D-luciferin Firefly). Such a moiety enables quantitative evaluation of heparanase activity via a suitable detecting equipment, e.g., a spectrophotometer, fluorometer or luminometer. A preferred quantitative colorimetric assay is the tetrazolium blue (an oxidative reagent) assay in which the reagents are reduced to a soluble colored formazan salt by the degraded substrate.

Thus, the present invention provides methodology which can be used to diagnose the presence or absence of the haemostatic disorder in a subject, such as a human. There is provided a variety of alternative techniques to perform the diagnosis which may be considered according to the special needs and availability of resources. The practice of this methodology, as illustrated in the Example section that follows, has conclusively shown that the present methodology provides a discriminative, reliable and accurate diagnosis approach.

The method of the present invention can be carried out at any laboratory capable of accessing the suitable reagents and detecting equipment. To facilitate testing, the present invention can be provided as a kit which includes all the necessary materials, reagents and instructions for use and interpretation of results.

Thus, according to another aspect of this invention there is provided a kit for diagnosing haemostatic disorders. The kit includes at least one container for holding at least one reagent for determining a level of heparanase expression or activity in a biological sample. The kit further includes packaging material which identifies the reagent or reagents for use in diagnosing haemostatic disorders.

According to one embodiment of this aspect of the invention the kit includes at least one container, preferably a microtiter plate, and at lease one heparanase specific antibody reagent. The antibody reagent may be provided as a working solution, or as a freeze-dried powder. The kit further includes a set of buffer solutions, a leaflet of instructions for use and a packaging material which identifies the kit for use in diagnosing haemostatic disorders.

Preferably the heparanase specific antibody is coupled to biotin or an enzyme, such as, but not limited to, horseradish peroxidase, alkaline phosphatase or β galactosidase.

Alternatively, the reagent includes an antibody binding protein that is coupled to an enzyme, such as, but not limited to, horseradish peroxidase, alkaline phosphatase or β galactosidase.

Procedures for coupling antibodies with enzymes are described in details in, for example “Using antibodies: A Laboratory Manual” [Ed Harlow, David Lane eds., Cold Spring Harbor Laboratory Press (1999)]. Anti-immunoglobulins coupled to enzymes are commercially available from biochemical reagent suppliers such as Sigma BioSciences.

In another embodiment of this aspect of the invention, the kit includes at least one substrate of the enzyme. Preferably, the substrate includes a detectable moiety that is chromogenic, fluorogenic or light emitting. For example, tetramethylbenzidine chromogenic substrate for horseradish peroxidase, or methylumbelliferyl phosphate fluorogenic substrate for alkaline phosphatase.

In a preferred embodiment of this aspect of the invention the kit comprises heparanase protein as a standard, a heparanase-specific monoclonal or polyclonal antibody prepared as described in U.S. Pat. No. 6,177,545, a biotinylated monoclonal or polyclonal antibody and a neutroavidin conjugated to a horseradish peroxidase enzyme. The kit also includes a TMB substrate solution, 0.05M barbonate/bicarbonate solution, PBS 0.05% Tween solution, PBS 1% and BSA 0.05% solution, SDS 1% solution, a 2M sulfuric acid solution, a standard 96 well microtiter plate (Nalge) and a packaging material identifying the kit and reagents for use in diagnosing haemostatic disorders. Further description of the methodology of implementing the use of the kit for determining the level of heparanase in biological samples is provided in the Examples section which follows

An alternative to an enzyme-linked immunoassay is to employ an antibody labeled with a detectable moiety, preferably, a radioactive, chromogenic, fluorogenic or light emitting moiety.

The enzymatic activity of heparanase may also be determined in a simple procedure which may be performed in the clinical laboratory equipped with standard bioanalytical equipment. While the quantitative analysis of heparanase by its enzymatic activity is generally considered to be less accurate as compared with an immunoassay analysis, it is relatively simple to perform and requires less costly consumable reagents. Therefore, enzymatic activity testing may be preferred over an immunoassay in situations such as where high throughput samples analyses are required and budget is tight. Thus, in order facilitate convenience of testing heparanase activity in practice the present invention provides a kit for the heparanase enzymatic activity testing.

Therefore, according to another embodiment of this aspect of the invention the kit includes at least one container, preferably a 96 well microplate, at lease one heparanase substrate which may be provided as a working solution, or freeze dried, contained in a vial. The kit further includes a set of buffer solutions, a leaflet of instructions and a packaging material which identifies the kit for use in diagnosing haemostatic disorders.

According another embodiment of this aspect of the invention the heparanase substrate is ECM, heparan sulfate proteoglycan, heparan sulfate, heparin or heparin-sepharose. Preferably, the substrate includes a detectable moiety selected from the group consisting of a chromogenic moiety, a fluorogenic moiety and a light emitting moiety.

In a preferred embodiment of this aspect of the invention the heparanase specific substrate is heparan sulfate. The kit further includes a tetrazolium blue solution (an oxidizing agent), buffer solutions, a 96 well microtiter plate (Nalge, USA), a leaflet of instructions for use and a packaging material identifying the kit for use in diagnosing haemostatic disorders.

Since treatment of haemostatic disorders often leads to serious, at times life threatening side effects, there is a need for an accurate monitoring methodology which will allow a physician to perform therapy adjustments according to the patients condition and severity of the disorder and to determine the dose and duration of treatment.

In addition, such accurate monitoring methodology of the level of a heparanase marker enables evaluating the efficacy of different treatments to prevent or to cure haemostatic disorders.

Thus, according to another aspect of the present invention, there is provided a method of determining the changes of a heparanase expression or activity in a biological sample obtained from a treated subject prior to, during or following the haemostatic disorder treatment, whereas the treatment may be either prophylactic, therapeutic or experimental.

The method according to this aspect of the present invention is effected by monitoring prior to, during or following treatment the heparanase levels of the treated individuals. The heparanase levels monitored serve as an indicator for the efficacy of treatment and thus can be used to adjust treatment

For example, monitoring heparanase levels in individuals treated with a thrombin inhibitor or an anticoagulant or an anti platelet drug (e.g., heparin, low molecular weight heparins, hirudin, PPACK, aspirin etc.) can serve to adjust dosages administered to the individual.

In conclusion, the present invention provides methods and kits utilizing heparanase as a diagnostic marker for haemostatic disorders. The heparanase marker is particularly advantageous because it is expressed in relatively large amounts from the onset of primary haemostasis throughout the progression of haemostatic conditions.

As shown in the Examples section below, there is a noted and substantial gap between the baseline level of heparanase found in group of healthy subjects and the level of heparanase found in patients with haemostatic disorders. Hence, heparanase is a useful, discriminative and reliable marker for diagnosing the presence, or absence of a haemostatic disorder or condition in individuals.

In addition, since heparanase is expressed from the onset and throughout progression a haemostatic condition, the level of heparanase can be used as a quantitative marker for diagnosing the severity of the haemostatic disorder/condition.

Finally, heparanase levels can be easily detected and quantitatively measured in biological fluid samples via a variety of techniques, thus enabling diagnosis of haemostatic disorders at any conventional clinical facility.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples

### EXAMPLES

Reference is now made to the following examples, which together with the above descriptions, illustrate the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Experimental Methods

Enzyme-linked immunosorbent assay (ELISA) was performed using a heparanase-specific monoclonal antibody (MAb) designated HP-117, prepared as described in U.S. Pat. No. 6,177,545B1. A standard 96-well microtiter plate was coated with the heparanase-specific MAb using a suspension of 10 ug MAb per ml of 0.05M carbonate/bicarbonate buffer, pH 9.6. The plate was washed four times with a PBS, 0.05% Tween solution. The plate was then blocked using a solution of PBS, 1% BSA, 0.05% Tween for 3-4 hrs at RT and then washed four times with the washing-buffer solution. Sample and standard aliquots were added to MAb-coated wells in duplicates, and were diluted, as indicated, in a solution of PBS, 1% BSA, 0.05% Tween and SDS, 0.1%. Recombinant human heparanase purified from Chinese hamster ovary (CHO) cells was used as a standard. Following application of samples, the plate was incubated overnight at 4° C. and then washed five times with the washing-buffer solution. Biotinylated αp45-heparanase polyclonal antibody (0.2 ug/ml in blocking-buffer solution), was added, the plate was incubated for 2 hrs at RT, and then washed five times with the washing-buffer solution. Neutroavidin Reagent conjugated to horseradish peroxidase (Pierce), diluted 1:5,000 in blocking-buffer solution was added to each well. The plate was incubated for 15 minutes at 37° C. and then washed five times with the washing-buffer solution. Substrate solution TMB (tetramethylbenzidine) was then added and the plate was incubated for additional 20-30 minutes at RT, then followed by the addition of a 2M Sulfuric Acid stop solution. Finally, Optical Density values were measured at 450 and 630 ηm wavelengths using a standard ELISA plate-reader.

### Example 1

### Comparing Levels of Heparanase in Plasma and Serum Samples, With or Without Thrombin

Blood samples were collected from two donors and were treated as follows:

(i) sample was added to EDTA-containing tube (plasma) and 1IU thrombin were added to sample;

(ii) sample was added to EDTA-containing tube (plasma) but no thrombin was added;

(iii) no EDTA was added (serum) and 1IU thrombin was added to sample; or

(iv) no EDTA was added (serum) and no thrombin was added to sample.

The treated samples were assayed using the ELISA procedure as described above. The results, summarized in Table 1, show that sera samples (without EDTA) and without thrombin, as compared with plasma samples (with EDTA, hence in vitro clotting was inhibited) and without thrombin, had substantially higher levels of heparanase, by 3.8 fold and 9.7 fold with sample 1 and 2, respectively. Similarly plasma samples (with EDTA) with thrombin (hence in vitro clotting was inhibited), as compared with plasma samples without thrombin, also had substantially higher levels of heparanase, by 3.8 fold and 7.9 fold with sample 1 and 2, respectively.

TABLE 1
Heparanase levels in blood samples treated with EDTA (serum),
without EDTA (plasma), with thrombin, or without thrombin.

Heparanase level (ng/ml)*

| Treatment | Patient 1 | Patient 2 |
|---|---|---|
| Serum | 112.80 | 96.70 |
| Serum + thrombin | 132.70 | 64.30 |
| Plasma | 30.10 | 10.00 |
| Plasma + thrombin | 115.50 | 78.50 |

*blood samples where diluted 1:100

### Example 2

### Comparing Levels of Heparanase in Plasma and Serum Samples, Kept at Room Temperature or Kept Frozen

Blood samples were collected from four donors and were treated as follows:

(i) sample was added to EDTA-containing tube (plasma) and sample was kept at room temperature;

(ii) sample was added to EDTA-containing tube (plasma) and sample was frozen at −80° C.;

(iii) no EDTA was added (serum) and sample was kept at room temperature; or

(iv) no EDTA was added (serum) and sample was kept was frozen at −80° C.

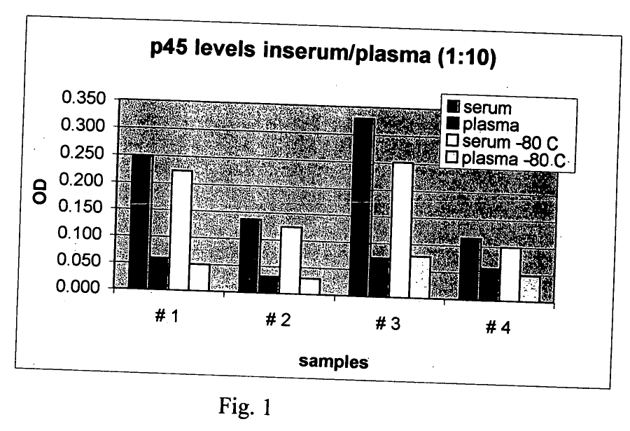

The treated samples were assayed using the ELISA procedure as described. The results, summarized in FIG. 1, show that sera samples (without EDTA) kept at room temperature, as compared with plasma samples (with EDTA, hence in vitro clotting was inhibited) and also kept at room temperature, had substantially higher levels of heparanase, by 3.8 fold on average. Similarly frozen sera samples (without EDTA), as compared with frozen plasma samples (with EDTA, hence in vitro clotting was inhibited), also had substantially higher levels of heparanase, by 3.4 fold on average. Thus, platelet activation and blood clotting increase the levels of heparanase in the blood.

### Example 3

### Measuring Levels of Heparanase in Plasma Samples Taken From Patients With or Without Haemostatic Disorders

Plasma samples were collected from patients and blindly tested for heparanase using the heparanase-specific ELISA procedure as described. Heparanase levels measured in healthy people (control group) are presented in Table 2. The results show that the level of plasma heparanase in healthy people was consistently less than 150 ng/ml. The one sample which exhibited higher than normal level of heparanase (greater than 250 ng/ml) was taken from a pregnant woman. Thus, freezing of samples does not affect the levels of heparanase in the specimen.

TABLE 2
Heparanase levels in plasma of healthy people

| | Heparanase Level |
|---|---|
| Control Number | (ng/ml) |
| 1 | >250 * |
| 2 | <100 |
| 3 | <150 |
| 4 | <100 |
| 5 | <100 |
| 6 | <100 |
| 7 | <150 |
| 8 | <50 |

* Sample was taken from a pregnant woman.

Levels of heparanase measured in samples of patients with various hematological disorders, are presented in Table 3, along with the respective clinical data of these patients. The data obtained herein show all of the samples which exhibited heparanase level higher than 150 ng/ml (15 samples) originated from patients with haemostatic disorders, [either deep vein thrombosis (DVT) or pulmonary embolism (PE), or their haemostatic disorder was not diagnosed]. On the other hand, in the 23 samples which exhibited heparanase levels of 150 ng/ml or lower cross reference with respective clinical data of these patients revealed that only one out of these 23 samples (4.3%) was taken from a patient with haemostatic disorder (# 32).

By comparison, the conventional diagnostic D-Dimer test detected very high levels of D-Dimer in 3 out of 9 patients (#3,10,31) which had not been diagnosed with a haemostatic disorder (false positive diagnosis), while failing to detect a haemostatic disorder in two patients one of which (#8) had DVT (false negative diagnosis).

TABLE 3
Heparanase and D-dimers levels in plasma of patients with various
hematological disorders

| | | | D-dimers^{a} | Heparanase | |
|---|---|---|---|---|---|
| Patient # | Sex | Age | (× 10²) | (ng/ml) | Diagnosis^{b} |
| 1 | F | 87 | | >600 | DVT + PE |
| 22 | F | 80 | I (10-20) | >400 | PE |
| 21 | M | 63 | | >250 | Lung carcinoma + DVT |
| 23 | F | 88 | H (20-40) | >300 | PE |
| 37 | M | 92 | | >200 | PE + DVT |
| 44 | F | 90 | | >500 | PE |
| 40 | F | 71 | | >150 | DVT |
| 45 | M | | | >250 | DVT |
| 49 | F | 77 | | >1500 | DVT |
| 32 | M | 31 | H (20-40) | >100 | DVT + superficial |
| | | | | | thrombophlebitis |
| 8 | M | 47 | L (2.5-5) | >150 | DVT |
| 42 | F | 89 | H (20-40) | >150 | Sepsis + recent arterial thrombosis |
| 48 | M | 80 | I (10-20) | >500 | Unknown |
| 38 | M | 83 | I (10-20) | >150 | Unknown |
| 12 | F | 95 | H (20-40) | >400 | Unknown |
| 39 | M | 75 | H (>40) | >150 | Unknown, high PT |
| 33 | M | 77 | H (20-40) | <50 | Unknown |
| 5 | M | 45 | I (10-20) | <25 | Liver metastases |
| 10 | F | | H (20-40) | <100 | Sepsis, pancreatic cancer, high PT |
| 26 | F | 72 | I (10-20) | <50 | Adrenal metastases from gastric |
| | | | | | carcinoma |
| 17 | F | 80 | | 0 | Myeloma |
| 19 | M | 67 | | 0 | Myeloma |
| 27 | F | | I (10-20) | <150 | Abdominal mass |
| 35 | M | 66 | | 0 | Treated Logret lymphoma |
| 7 | M | 24 | | 0 | ITP |
| 11 | | 67 | | <50 | ITP |
| 13 | M | | | <50 | ITP |
| 29 | F | | | <50 | ITP |
| 30 | F | | | 0 | ITP |
| 41 | F | 35 | | 0 | Thrombocytopenia for unknown |
| | | | | | reason |
| 28 | F | | | 0 | Thrombocytopenia for unknown |
| | | | | | reason |
| 20 | M | 47 | L (5-10) | <150 | Endocarditis, high PT |
| 18 | M | | I (10-20) | <50 | Sepsis |
| 31 | F | 80 | H (20-40) | <150 | DIC |
| 24 | F | 68 | I (10-20) | 0 | Pneumonia |
| 16 | M | | | 0 | Myocardial infaction |
| 3 | M | 55 | H (>40) | <100 | Sepsis (Erysepelas), high PT |
| 4 | M | 88 | I (10-20) | 0 | Septic shock |

^{a}H = high L = low I = intermediate
^{b}DVT = deep vein thrombosis;
PE = pulmonary embolism;
DIC = disseminated intravascular coagulation;
ITP = autoimmune thrombocytopenia

Heparanase mediated diagnosis of haemostatic disorders provides several distinct advantages over prior art techniques since:

(i) heparanase is expressed during the primary coagulation process as well as during the pathogenesis process, and therefore the heparanase expression is indicative of presence, absence or progression of any kind of haemostatic disorder;

(ii) heparanase is an enzyme, and therefore the heparanase activity can be measured specifically and accurately via a wide range of immunoassays, or alternatively, by enzymatic activity assays; and

(iii) heparanase is expressed in a relatively large amount in subjects afflicted with haemostatic disorders, and therefore heparanase level may be conveniently and sensitively measured using standard diagnostic tools (e.g., ELISA plate reader, fluorometer, luminometer) which are readily available in most conventional clinical laboratories.

Hence, heparanase is a unique and accurate marker for diagnosing haemostatic disorders. Heparanase may be measured non-invasively in a biological fluid sample, conveniently, rapidly, sensitively and accurately using standard diagnostic techniques.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

What is claimed is:

1. A method of determining a presence, absence, or severity of a haemostatic disorder in a subject, the method comprising determining a level of heparanase expression or activity in a biological sample obtained from the subject and based on said level of said heparanase expression or activity determining the presence, absence, or severity of the of haemostatic disorder in the subject.

2. The method of claim 1, further comprising comparing said level of heparanase expression or activity in said biological sample obtained from the subject to that of a normal individual and/or an individual suffering from the haemostatic disorder.

3. The method of claim 1, wherein said biological sample is a biological fluid selected from the group consisting of blood, plasma, cerebral fluid, and urine.

4. The method of claim 1, wherein said determining said level of heparanase expression in said biological sample is effected via an immunoassay.

5. The method of claim 4, wherein said immunoassay is selected from the group consisting of enzyme-linked immunosorbent assay (ELISA), a radioimmunoassay, an immunofluorescence assay, and a light emission immunoassay.

6. The method of claim 1, wherein said determining said level of heparanase activity in said biological sample is effected by using an heparanase substrate.

7. The method of claim 6, wherein said heparanase substrate is selected from the group consisting of heparan sulfate proteoglycans, heparan sulfate, heparin, and heparin-sepharose.

8. The method of claim 6, wherein said heparanase substrate includes a detectable moiety selected from the group consisting of a chromogenic moiety, a fluorogenic moiety and a light emitting moiety.

9. A kit for diagnosing a haemostatic disorder comprising at least one container including at least one reagent for determining a level of heparanase expression or activity in a biological sample, and packaging material identifying said at least one reagent for use in diagnosing the haemostatic disorder.

10. The kit of claim 9, wherein said reagent includes a heparanase specific antibody.

11. The kit of claim 10, wherein said heparanase specific antibody is coupled to an enzyme.

12. The kit of claim 11, wherein said reagent includes a substrate of said enzyme.

13. The kit of claim 12, wherein said substrate includes a chromogenic, fluorogenic or light emitting moiety.

14. The kit of claim 9, wherein said reagent includes a heparanase substrate.

15. The kit of claim 14, wherein said heparanase substrate is selected from the group consisting of heparan sulfate proteoglycans, heparan sulfate, heparin, and heparin-sepharose.

16. The kit of claim 14, wherein said heparanase substrate includes a detectable moiety selected from the group consisting of a chromogenic moiety, a fluorogenic moiety, and a light emitting moiety.

17. A method of monitoring an effectiveness of a haemostatic disorder treatment, comprising determining a level of heparanase expression or activity in biological samples obtained from a subject prior to, during and/or following treatment for the haemostatic disorder and based on said level of said heparanase expression or activity, monitoring the effectiveness of the haemostatic disorder treatment.

18. The method of claim 17, wherein the haemostatic disorder treatment is effected by administering to the individual a pharmaceutical composition.

19. The method of claim 17, wherein said pharmaceutical composition includes an active ingredient selected from the group consisting of a thrombin inhibitor, an anticoagulant, a platelet aggregation inhibitor and a heparanase inhibitor.
